(19) United States
(12) Patent Application Publication
(10) Pub. No.: US 2023/0248667 A1
(43) Pub. Date: Aug. 10, 2023

(54) EXTENDED RELEASE COMPOSITIONS OF PSEUDOEPHEDRINE OR ITS PHARMACEUTICALLY ACCEPTABLE SALT THEREOF

(71) Applicant: Aurobindo Pharma Ltd, Hyderabad (IN)

(72) Inventors: Soumyajit Behera, Hyderabad (IN); Satheesh Kumar Gajjala, Hyderabad (IN); Nilesh Bhandari, Hyderabad (IN); Nagaprasad Vishnubhotla, Hyderabad (IN); Sivakumaran Meenakshisunderam, Hyderabad (IN)

(73) Assignee: Aurobindo Pharma Ltd, Hyderabad (IN)

(21) Appl. No.: 18/105,795

(22) Filed: Feb. 3, 2023

(30) Foreign Application Priority Data
Feb. 4, 2022 (IN) 202241006098

(51) Int. Cl. A61K 31/137; A61K 31/495; A61K 9/28

(52) U.S. Cl. CPC A61K 31/137; A61K 31/495; A61K 9/2893; A61K 9/2866; A61K 9/282

(57) ABSTRACT

This invention relates to dosage forms comprising pseudoephedrine or a salt thereof, for extended-release up to 24 hours, and process of making such dosage forms. This invention provides a non-osmotic extended-release dosage form comprising pseudoephedrine hydrochloride which can be administered to patients who need and/or desire a decongestant medication up to 24 hours. This invention also relates to dosage forms comprising pseudoephedrine or a salt thereof and cetirizine or levocetirizine or its salts thereof for extended-release up to 24 hours, and process of making such dosage forms.

### CROSS REFERENCE TO RELATED APPLICATIONS

This application claims priority from an Indian Patent Application IN 202241006098 filed on Feb. 4, 2022

### FIELD OF THE INVENTION

The present invention relates to dosage forms comprising pseudoephedrine or a salt thereof, for extended-release up to 24 hours, and process of making such dosage forms.

This invention provides a non-osmotic extended-release dosage form comprising pseudoephedrine hydrochloride which can be administered to patients who need and/or desire a decongestant medication up to 24 hours.

BACKGROUND OF THE INVENTION

Pseudoephedrine was first known as a natural substance that occurs in shrubs of the Ephedra genus, which grow worldwide. Pseudoephedrine is used to relieve nasal or sinus congestion caused by the common cold, sinusitis, and hay fever and other respiratory allergies. It is also used to relieve ear congestion caused by ear inflammation or infection. SUDAFED® 24 HOUR (Pseudoephedrine hydrochloride) extended release tablet 240 mg was approved on Dec. 15, 1992 to J AND J CONSUMER INC in the USA Market. Inactive ingredients of SUDAFED® 24 HOUR tablet are cellulose triacetate, hydroxypropylcellulose, hypromellose, iron oxide, magnesium stearate, microcrystalline cellulose, polyethylene glycol, polysorbate 80, povidone, propylene glycol, shellac, sodium chloride, titanium dioxide.

SUDAFED® 24 HOUR tablets are osmotic tablets, wherein a drug-containing tablet comprising of a osmotic agent, sodium chloride is surrounded by a semipermeable membrane (cellulose acetate and hydroxypropyl cellulose in a specific ratio of 3:1) and one or more lamina with or without drug. A laser drilled opening in the tablet acts as the vent for drug delivery. In the gastrointestinal tract, water enters the tablet and a contained osmotic agent swells to exert the pressure required for the controlled release of the drug through the opening. There are various modifications that are available for this technology. However, formulating an osmotic drug delivery system presents challenges, such as the specialized equipment required for laser drilled holes and multi-step processing for making tablet layers and coatings and time consuming for large-scale production of such osmotic pseudoephedrine hydrochloride extended release tablet 240 mg. U.S. Pat. No. 4,801,461 of Alza corporation, discloses such osmotic pseudoephedrine hydrochloride extended release tablet comprising: (a) a compartment (b) a dosage amount of about 160 to 200 mg of a member selected from the group consisting of pseudoephedrine and its therapeutically acceptable salts in the compartment; (c) a wall comprising at least in part from 70 to 85 weight percent of a cellulose acetate comprising an acetyl content of 35% to 43.5% and from 15 to 30 weight percent hydroxypropylcellulose, which wall is permeable to the passage of an external fluid, surrounds and defines the compartment and aids in protecting pseudoephedrine present in the compartment from a premature exposure to the environment of use; (d) at least one passageway in the wall for connecting the compartment with the exterior of the dosage form; (e) a lamina comprising 55 to 65 mg of a member selected from the group consisting of pseudoephedrine and its therapeutically acceptable salts in laminar arrangement with the exterior of the wall; and, (f) wherein, when the dosage form is in operation, the dosage form administers the pseudoephedrine immediately from the lamina and at a metered release rate per unit time from the compartment.

There is a long-felt need for making simple non-osmotic pseudoephedrine hydrochloride extended release tablet 240 mg. Since, pseudoephedrine hydrochloride is a Class I (highly soluble & highly permeable) drug compound according to biopharmaceutics classification system (BCS), and after administration there is always a risk of immediately releasing all of its medication. It is very difficult to design a non-osmotic pseudoephedrine hydrochloride extended release tablet 240 mg, which can have comparative drug dissolution and/or drug bioavailability to that of drug dissolution and/or drug bioavailability of SUDAFED® 24 HOUR (pseudoephedrine hydrochloride) extended release tablet 240 mg.

Several attempts were made earlier for example US20100260842 A1 of Dr. Reddy's Laboratories Ltd., discloses a controlled release formulation comprising pseudoephedrine or a salt thereof, comprising: I. a core comprising: a) 10 to 40 percent by weight of pseudoephedrine or a salt thereof; b) 5 to 30 percent by weight of a water soluble polymer; c) 1 to 10 percent by weight of a binder; d) 15 to 40 percent by weight of a water insoluble filler; and e) optionally, one or more other pharmaceutical excipients; and II. a coating comprising: a) 1 to 15 percent by weight of pseudoephedrine or a salt thereof; b) 1 to 25 percent by weight of a combination of a water soluble polymer and a water insoluble polymer; and c) 0.1 to 15 percent by weight of a water insoluble plasticizer. It also discloses a controlled release formulation comprising pseudoephedrine or a salt thereof, comprising: I. a core containing about 50-90 percent of a total amount of pseudoephedrine in the formulation, comprising: f) 10 to 40 percent by weight of pseudoephedrine or a salt thereof; g) 5 to 30 percent by weight of a water soluble polymer; h) 1 to 10 percent by weight of a binder; i) 15 to 40 percent by weight of a water insoluble filler; and j) optionally, one or more other pharmaceutical excipients; and II. a coating containing about 10-50 percent of a total amount of pseudoephedrine in the formulation, comprising: c) 1 to 15 percent by weight of pseudoephedrine or a salt thereof; d) 1 to 25 percent by weight of a combination of a water soluble polymer and a water insoluble polymer; and e) 0.1 to 15 percent by weight of a water insoluble plasticizer.

It was found that water insoluble polymers especially ethyl cellulose is not a suitable polymer for extended release coating on to the tablet core as there are problems related to scalability, reproducibility relating to desired and consistent dissolution profile as per the drug product specifications.

It is generally known in state of art that making a non-osmotic pseudoephedrine hydrochloride extended release tablet 240 mg is a very difficult task due to one or more below listed challenges:—
1. High drug solubility & high drug permeability,
2. Selection/choice of excipients & its concentrations (for e.g. extended release polymers),
3. Ratio of excipients [for e.g. ratio of extended release polymers to plasticizer(s) and pore former (s)],
4. Design of non-osmotic extended release tablet (matrix type drug release mechanism or membrane type drug release mechanism or Combinations of matrix & membrane drug release mechanisms),
5. The composition of “functional coating” for e.g. selection/choice of rate controlling polymer(s), plasticizer(s), pore-former(s) and ratio thereof.
6. Processes and/or process parameters of making non-osmotic extended release tablet, and
7. Acceptable controlled drug release profile up to 24 hours, for e.g. an immediate release phase followed by drug delivery at a controlled rate over a period of time, preferably up to 24 hours.

### SUMMARY OF INVENTION

The present invention relates to non-osmotic extended-release pharmaceutical formulations comprising pseudoephedrine or its pharmaceutical salts, having an immediate release phase, followed by drug delivery at a controlled rate over a period of time, preferably up to 24 hours.

One aspect of the present invention relates to a non-osmotic extended release formulation comprising pseudoephedrine or a salt thereof, comprising:

I. a core comprising: a) about 60% to 90% of pseudoephedrine or a salt thereof by total weight of pseudoephedrine or a salt thereof; b) a water soluble extended release polymer; and c) one or more other pharmaceutical excipients;

II. optional seal coat;

III. extended-release coating comprising: cellulose acetate, at least one plasticizer, at least one pore-former;

IV. optional sub coat; and

V. an immediate release coating comprising about 10 to 40% of pseudoephedrine or a salt thereof by total weight of pseudoephedrine or a salt thereof, and one or more other pharmaceutical excipients.

In another aspect of the present invention relates to a non-osmotic extended release formulation comprising pseudoephedrine or a salt thereof and cetirizine or levocetirizine or a salt thereof, comprising:

I. a core comprising: a) about 60% to 90% of pseudoephedrine or a salt thereof by total weight of pseudoephedrine or a salt thereof; b) a water soluble extended release polymer; and c) one or more other pharmaceutical excipients;

II. optional seal coat;

III. extended-release coating comprising: cellulose acetate, at least one plasticizer, at least one pore-former;

IV. optional sub coat; and

V. an immediate release coating comprising about 10 to 40% of pseudoephedrine or a salt thereof by total weight of pseudoephedrine or a salt thereof, and about 1 to 10% of cetirizine or levocetirizine or a salt thereof, and one or more other pharmaceutical excipients.

Another aspect of the present invention relates to a non-osmotic extended release formulation comprising pseudoephedrine or a salt thereof, comprising:

I. a core comprising: a) about 75% of pseudoephedrine or a salt thereof by total weight of pseudoephedrine or a salt thereof; b) hydroxypropylmethyl cellulose; and c) one or more other pharmaceutical excipients;

II. optional seal coat;

III. extended-release coating comprising: cellulose acetate, at least one plasticizer, at least one pore-former;

IV. optional sub coat; and

V. an immediate release coating comprising about 25% of pseudoephedrine or a salt thereof by total weight of pseudoephedrine or a salt thereof and hydroxypropylmethyl cellulose.

Another aspect of the present invention relates to a non-osmotic extended release formulation comprising pseudoephedrine or a salt thereof, wherein an amount of pseudoephedrine or a salt thereof released from the extended release tablet composition is from about 20% to 35% at 2 hours, from about 40% to 75% at 8 hours, from about 65% to 90% at 12 hours and not less than 85% at 24 hours.

In yet another aspect of the present invention relates to a non-osmotic extended release formulation comprising pseudoephedrine or a salt thereof, having a comparative bioavailability profile with that of commercially available pseudoephedrine hydrochloride 240 mg extended release tablet.

In yet another aspect of the present invention relates to a non-osmotic extended release formulation comprising pseudoephedrine or a salt thereof, indicated for the relief of symptoms associated with nasal congestion in adults and children 12 years of age and older.

### DETAILED DESCRIPTION OF THE INVENTION

The present invention relates to non-osmotic extended-release pharmaceutical formulations comprising pseudoephedrine or its pharmaceutical salts, having an immediate release phase, followed by drug delivery at a controlled rate over a period of time, preferably up to 24 hours.

As used herein, the term “pseudoephedrine” includes the compound pseudoephedrine and any of its pharmaceutically acceptable salts, polymorphs, solvates, esters, hydrates, enantiomers, racemates, and mixtures thereof. Preferably, it is pseudoephedrine hydrochloride.

As used herein, the term “controlled release (CR)” or “extended release (ER)” indicates a formulation that releases drug in a manner different from that of an immediate release dosage form (in which drug release commences promptly in the stomach after oral administration, and continues at a rapid rate as the dosage form disintegrates), such as providing a drug release that is delayed for a desired time period after administration, and/or a drug release that occurs during a desired time period after administration. The terms ‘sustained’, ‘extended’, ‘delayed’, ‘modified’, or ‘prolonged’ are included within the term “controlled,” and as applied include any drug delivery system which achieves the release of drug after and/or over a desired period of time, e.g., providing dosage forms that can be administered every 8 hours, 12 hours, 24 hours, etc. The drug delivery systems include those such as modified-release matrix cores, modified-release matrix cores coated with a release-modifying coating, enteric coated dosage forms, dosage forms surrounded by a release-modifying coating, gastro-retentive dosage forms, muco-adhesive dosage forms, osmotic release dosage forms, etc. Immediate release (IR) pseudoephedrine products typically require dosing at 4-6 hour intervals, to provide the desired therapeutic effect.

The terms “dosage form” or “composition” or “formulation” as used herein relate to solid orally administered units comprising a drug and one or more pharmacologically inert excipients, such as tablets, mini-tablets, caplets, capsules, granules, pills, powders, microparticles, nanoparticles, etc.

The term “functional coating” relates to any type of coating that modifies the release of pseudoephedrine or its salts from a dosage form. The rate-controlling polymers or extended release polymers or its combinations with have a significant role in modification of the release of pseudoephedrine or its salts from a dosage form.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The term “core” as used herein is defined to mean a particle or a solid vehicle, into which, or onto which, pseudoephedrine is uniformly or non-uniformly dispersed. A drug-containing core will desirably be formed by methods and materials well known in the art, such as for example by compressing, fusing, or extruding pseudoephedrine alone or together with at least one pharmaceutically acceptable excipient, or coating a pseudoephedrine composition onto a pharmacologically inert substrate.

In an embodiment, the present invention provides a non-osmotic extended release formulation comprising pseudoephedrine or a salt thereof, comprising:

I. a core comprising: a) about 60% to 90% of pseudoephedrine or a salt thereof by total weight of pseudoephedrine or a salt thereof; b) a water soluble extended release polymer; and c) one or more other pharmaceutical excipients;

II. optional seal coat;

III. extended-release coating comprising: cellulose acetate, at least one plasticizer, at least one pore-former;

IV. optional sub coat; and

V. an immediate release coating comprising about 10% to 40% of pseudoephedrine or a salt thereof by total weight of pseudoephedrine or a salt thereof, and one or more other pharmaceutical excipients.

For example core is a compressed tablet comprising about 180 mg of pseudoephedrine hydrochloride, hydroxypropylmethyl cellulose, and one or more other pharmaceutical excipients selected from diluents, binders, glidants, lubricants and others.

Exemplary diluents include, but not limited to, lactose, microcrystalline cellulose, sucrose, mannitol, xylitol, erythritol, sorbitol, maltitol, calcium citrate, calcium phosphate, and calcium aluminometasilicate, calcium carbonate, calcium phosphate, dicalcium phosphate, calcium sulfate, dextrin/dextrates, maltodextrin, and compressible sugars or mixtures thereof.

Exemplary lubricants include magnesium stearate, zinc stearate, calcium stearate, talc, carnauba wax, stearic acid, palmitic acid, sodium stearyl fumarate, sodium lauryl sulfate, glyceryl palmitostearate, palmitic acid, myristic acid and hydrogenated vegetable oils and fats.

Exemplary glidants include talc, colloidal silicon dioxide, silicic acid, calcium silicate, magnesium carbonate, magnesium oxide, magnesium silicate, starch, castor wax and the like.

Preferably, core tablet is a compressed tablet comprising about 180 mg of pseudoephedrine hydrochloride, hydroxypropylmethyl cellulose, lactose monohydrate, microcrystalline cellulose, colloidal silicon dioxide, stearic acid. The process of making such compressed tablet can be according to standard techniques of tableting viz; direct compression or wet granulation or dry granulation. The direct compression is being preferred.

Exemplary water soluble extended release polymers include, but are not limited to, hydroxypropyl methyl cellulose (HPMC), sodium alginate, hydroxypropyl cellulose, methyl cellulose, carboxymethyl cellulose, or mixtures thereof. Preferred water soluble extended release polymer is hydroxypropyl methyl cellulose.

Exemplary plasticizers, include but are not limited to, dibutyl sebacate, triacetin, diethyl phthalate, tributyl sebacate, glycerin, triacetin, and triethyl citrate or mixtures thereof.

Exemplary pore formers, include but are not limited to, polyethylene glycols, hydroxypropylmethyl cellulose, hydroxypropyl cellulose, glucose, fructose, mannitol, sorbitol, dextran, polyvinylpyrrolidone, polypropylene glycols or mixtures thereof.

Seal coat or sub coat can be formed by conventional coating techniques with various useful film-forming agents include, but are not limited to, cellulose or its derivatives including soluble alkyl- or hydroalkyl-cellulose derivatives such as methylcelluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates, and methylhydroxypropylcellulose phthalates, polyvinylacetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, poly acrylic acids, polyvinylalcohols, polyvinylacetates, polyvinylpyrrolidones, polymethacrylates and derivatives thereof (Eudragit.™.), chitosan and derivatives thereof, shellac and derivatives thereof, and waxes and fat substances. Instant coating materials that are commercially available in the market also can be used such as OPADRY coating materials including Opadry AMB II 88A180040 white which contains polyvinyl alcohol, talc, titanium dioxide, glycerylmonocaprylocaprate, sodium lauryl sulfate.

In an embodiment, the present invention provides a non-osmotic extended release tablet comprising pseudoephedrine hydrochloride, comprising:

I. a tablet core comprising: a) about 60% to 90% of pseudoephedrine hydrochloride by total weight of pseudoephedrine hydrochloride; b) hydroxypropylmethyl cellulose; and c) one or more other pharmaceutical excipients;

II. optional seal coat;

III. extended release coating comprising: cellulose acetate, dibutyl sebacate, polyethylene glycol;

IV. optional sub coat; and

V. an immediate release coating comprising about 10% to 40% of pseudoephedrine hydrochloride by total weight of pseudoephedrine hydrochloride, and one or more other pharmaceutical excipients.

In an embodiment, the present invention provides a non-osmotic extended release tablet comprising pseudoephedrine hydrochloride, comprising:

I. a tablet core comprising: a) about 60% to 90% of pseudoephedrine hydrochloride by total weight of pseudoephedrine hydrochloride; about 40% to 60% of hydroxypropylmethyl cellulose; and c) one or more other pharmaceutical excipients;

II. seal coat comprising hydroxypropylmethyl cellulose;

III. extended release coating comprising: cellulose acetate, dibutyl sebacate, polyethylene glycol;

IV. sub coat comprising hydroxypropylmethyl cellulose; and

V. an immediate release coating comprising about 10 to 40% of pseudoephedrine hydrochloride by total weight of pseudoephedrine hydrochloride, and one or more other pharmaceutical excipients.

In an embodiment, the present invention provides a non-osmotic extended release formulation comprising pseudoephedrine or a salt thereof, comprising:

I. a core comprising: a) about 60% to 90% of pseudoephedrine or a salt thereof by total weight of pseudoephedrine or a salt thereof; b) a water soluble extended release polymer; and c) one or more other pharmaceutical excipients;

II. optional seal coat;

III. extended-release coating comprising: cellulose acetate, at least one plasticizer, at least one pore-former in the weight ratio of about 1:0.1:0.1 to 1:1.5:1.5;

IV. optional sub coat; and

V. an immediate release coating comprising about 10% to 40% of pseudoephedrine or a salt thereof by total weight of pseudoephedrine or a salt thereof, and one or more other pharmaceutical excipients.

In another embodiment, the present invention provides a non-osmotic extended release tablet comprising:

I. a core comprising: a) about 180 mg of pseudoephedrine hydrochloride; b) hydroxypropylmethyl cellulose; and c) one or more other pharmaceutical excipients;

II. optional seal coat;

III. extended release coating comprising: about 50% to 75% weight of cellulose acetate, and about 25% to 50% weight mixture of dibutyl sebacate and polyethylene glycol, by total weight of extended release coating composition, wherein the weight ratio of cellulose acetate, dibutyl sebacate and polyethylene glycol is from about 1:0.1:0.1 to 1:1:1;

IV. optional sub coat; and

V. an immediate release coating comprising about 60 mg of pseudoephedrine hydrochloride and hydroxypropylmethyl cellulose.

In yet another embodiment, the present invention provides a non-osmotic extended release tablet comprising

I. a core comprising: a) about 180 mg of pseudoephedrine hydrochloride; b) hydroxypropylmethyl cellulose; and c) one or more other pharmaceutical excipients;

II. optional seal coat;

III. extended release coating comprising: about 50% to 75% weight of cellulose acetate, and about 25% to 50% weight mixture of dibutyl sebacate and polyethylene glycol, by total weight of extended release coating composition, wherein the weight ratio of cellulose acetate, dibutyl sebacate and polyethylene glycol is about 1:0.15:0.3;

IV. optional sub coat; and

V. an immediate release coating comprising about 60 mg of pseudoephedrine hydrochloride and hydroxypropylmethyl cellulose.

In yet another embodiment, the present invention provides a non-osmotic extended release tablet comprising

I. a core comprising: a) about 180 mg of pseudoephedrine hydrochloride; b) hydroxypropylmethyl cellulose; and c) one or more other pharmaceutical excipients;

II. optional seal coat;

III. extended release coating comprising: about 50% to 75% weight of cellulose acetate, and about 25% to 50% weight mixture of dibutyl sebecate and polyethylene glycol, by total weight of extended release coating composition, wherein the weight ratio of cellulose acetate, dibutyl sebecate and polyethylene glycol is from about 1:0.1:0.1 to 1:0.3:0.5;

IV. optional sub coat; and

V. an immediate release coating comprising about 60 mg of pseudoephedrine hydrochloride and hydroxypropylmethyl cellulose; wherein an amount of pseudoephedrine hydrochloride released from the extended release tablet composition is from about 20% to 35% at 2 hours, from about 40% to 75% at 8 hours, from about 65% to 90% at 12 hours and not less than 85% at 24 hours.

In yet another embodiment, the present invention provides a non-osmotic extended release tablet comprising:

I. a core comprising: a) about 180 mg of pseudoephedrine hydrochloride; b) hydroxypropylmethyl cellulose; and c) one or more other pharmaceutical excipients;

II. optional seal coat;

III. extended release coating comprising: about 50% to 75% weight of cellulose acetate, and about 25% to 50% weight mixture of dibutyl sebecate and polyethylene glycol, by total weight of extended release coating composition, wherein the weight ratio of cellulose acetate, dibutyl sebecate and polyethylene glycol is from about 1:0.1:0.1 to 1:0.3:0.5;

IV. optional sub coat; and

V. an immediate release coating comprising about 60 mg of pseudoephedrine hydrochloride and hydroxypropylmethyl cellulose; wherein the extended release tablet has a comparative bioavailability profile with that of bioavailability profile of commercially available pseudoephedrine hydrochloride 240 mg extended release tablet.

In an embodiment, the present invention provides a non-osmotic extended release tablet comprising pseudoephedrine hydrochloride and cetirizine or levocetrizine or its salts thereof, comprising:

I. a tablet core comprising: a) about 60% to 90% of pseudoephedrine hydrochloride by total weight of pseudoephedrine hydrochloride; hydroxypropylmethyl cellulose; and c) one or more other pharmaceutical excipients;

II. seal coat comprising hydroxypropylmethyl cellulose;

III. extended release coating comprising: cellulose acetate, at least one plasticizer, at least one pore-former;

IV. sub coat comprising hydroxypropylmethyl cellulose; and

V. an immediate release coating comprising 10 to 40% of pseudoephedrine hydrochloride by total weight of pseudoephedrine hydrochloride, and about 1 to 10% of cetirizine or levocetrizine or its salts thereof, and one or more other pharmaceutical excipients.

In yet another embodiment, the present invention provides a non-osmotic extended release formulation comprising pseudoephedrine or a salt thereof, indicated for the relief of symptoms associated with nasal congestion in adults and children 12 years of age and older.

The following examples are intended to serve as illustrations of the present invention only and do not restrict the scope of the invention in any manner whatsoever.

### EXAMPLES

### Example 1

Non-osmotic extended release pseudoephedrine hydrochloride tablets 240 mg (matrix tablet with combination of HPMC polymers).

| Ingredients | Quantity/tablet (mg) |
|---|---|
| Pseudoephedrine HCI | 240.00 |
| Hypromellose K 100M | 380.00 |
| Hypromellose E4M | 100.00 |
| Colloidal silicon dioxide | 4.00 |
| Stearic acid | 8.00 |
| Total | 732.00 |

Brief manufacturing process:
1. Pseudoephedrine hydrochloride and other excipients (except stearic acid) were sifted separately in a suitable sieve and blended for appropriate time to get homogeneous unlubricated blend.
2. Blend of Step-1 was lubricated with stearic acid and compressed into tablets.

### Example 2

Non-osmotic extended release pseudoephedrine hydrochloride tablets 240 mg (matrix tablet with combination of Hypromellose K100M in core & extended release coating membrane with a mixture of ethyl cellulose: Hypromellose—50:50)

| | |
|---|---|
| Ingredients | Quantity/tablet (mg) |

Core tablet

| | |
|---|---|
| Pseudoephedrine HCI | 240.00 |
| Hypromellose K 100M | 195.00 |
| Microcrystalline cellulose | 17.00 |
| Colloidal silicon dioxide | 4.00 |
| Stearic acid | 8.00 |
| Sub-total | 464.00 |

Extended release coating

| | |
|---|---|
| Ethyl Cellulose 100 cps (EC) | 41.76 |
| Hypromellose E5 LV | 41.76 |
| Isopropyl alcohol/Dichloromethane | Q.S. |
| Purified Water | Q.S. |
| Total | 547.52 |

Brief manufacturing process:

1. Pseudoephedrine hydrochloride core tablets were prepared by direct compression like example 1 process.

2. About 18% w/w of extended release coating were applied to core tablets of step 1.

### Example 3

Non-osmotic extended release pseudoephedrine hydrochloride tablets 240 mg (matrix tablet with ethyl cellulose and hypromellose in core & extended release coating membrane with cellulose acetate).

| | |
|---|---|
| Ingredients | Quantity/tablet (mg) |

Core tablet prepared by Top-spray granulation

| | |
|---|---|
| Pseudoephedrine HCI | 240.00 |
| Ethyl cellulose 10 cps | 48.00 |
| Ethanol 96% | Q.S. |
| Purified water | Q.S. |
| HPMC K100M DC2 | 100.00 |
| Colloidal silicon dioxide | 4.00 |
| Stearic acid | 8.00 |
| Sub-total | 400.00 |

Seal Coating

| | |
|---|---|
| Opadry AMB II 88A180040 | 14.00 |
| Sub-total | 414.00 |

Extended release coating

| | |
|---|---|
| Cellulose acetate with suitable coating aids | 24.84 |
| (selected one plasticizer, atleast one pore- | |
| former, Acetone & purified water) | |
| Total | 438.84 |

Brief manufacturing process:

1. Blend was prepared by spraying hydro alcoholic solution of ethyl cellulose sprayed on to pseudoephedrine hydrochloride (top-spray technique).

2. Dried granules of step-1 were blended with HPMC, colloidal silicon dioxide and lubricated with stearic acid.

3. Lubricated blend of step-2 compressed into core tablets.

4. Core tablets of step-3 were seal coated with aqueous dispersion of Opadry AMB II.

5. Extended release coating was applied to core tablets of step 4.

### Example 4

Non-osmotic extended release pseudoephedrine hydrochloride tablets 240 mg (matrix tablet with HPMC K100M & 240 mg of pseudoephedrine hydrochloride in core & 4.5% extended release membrane coating with cellulose acetate)

| | |
|---|---|
| Ingredients | Quantity/tablet (mg) |

Core tablet

| | |
|---|---|
| Pseudoephedrine HCI | 240.00 |
| Hypromellose K 100M | 190.50 |
| Lactose Monohydrate | 4.50 |
| Microcrystalline cellulose | 17.00 |
| Colloidal silicon dioxide | 4.00 |
| Stearic acid | 8.00 |
| Sub-Total | 464.00 |

Seal Coating

| | |
|---|---|
| Opadry AMB II 88A180040 white | 16.24 |
| Purified Water | Q.S. |
| Sub-total | 480.240 |

Extended release coating

| | |
|---|---|
| Cellulose acetate with suitable coating | 21.611 |
| aids (selected one plasticizer, atleast | |
| one pore-former, Acetone & purified | |
| water) | |
| Sub-total | 501.851 |

Film Coating

| | |
|---|---|
| Opadry AMB II 88A180040 white | 20.07 |
| Purified Water | Q.S. |
| Total | 521.92 |

Brief manufacturing process:

1. Pseudoephedrine hydrochloride core tablets were prepared by direct compression like example 1 process.

2. Seal coating: Aqueous dispersion of opadry AMB II white 88A180040 was sprayed onto the core tablets.

3. Extended release coating: Hydroalcoholic dispersion of Cellulose acetate was sprayed onto the seal coated tablets.

4. Film coating: Aqueous dispersion of opadry AMB II white 88A180040 was sprayed onto the extended release tablets.

### Example 5

Non-osmotic extended release Pseudoephedrine hydrochloride tablets 240 mg (matrix tablet with HPMC K100M & 75% pseudoephedrine hydrochloride in core, 25% pseudoephedrine in immediate release coating & extended release coating with cellulose acetate)

| | |
|---|---|
| Ingredients | Quantity/tablet (mg) |

Core tablet

| | |
|---|---|
| Pseudoephedrine HCI | 180.000 |
| Hypromellose K 100M | 157.875 |
| Lactose Monohydrate | 3.375 |
| Microcrystalline cellulose | 12.750 |
| Colloidal silicon dioxide | 3.000 |
| Stearic acid | 6.000 |
| Sub-total | 363.00 |

Seal coating

| | |
|---|---|
| Opadry AMB II 88A180040 white | 12.705 |
| Purified Water | Q.S. |
| Sub-total | 375.705 |

Extended release coating

| | |
|---|---|
| Cellulose acetate with suitable coating aids | 30.056 |
| dibutyl sebacate and polyethylene glycol, in | |
| a ratio of 1:0.15:0.3 | |
| Sub-total | 405.761 |

Sub coating

| | |
|---|---|
| Hypromellose | 12.173 |
| Purified Water | Q.S. |
| Sub-total | 417.934 |

Immediate release drug loading

| | |
|---|---|
| Pseudoephedrine HCI | 60.00 |
| Hypromellose | 10.00 |
| Purified water | Q.S. |
| Sub-Total | 487.934 |

Film coating

| | |
|---|---|
| Opadry AMB II 88A180040 white 19.517 | |
| Purified Water | Q.S. |
| Total | 507.451 |

Brief manufacturing process:

1. Pseudoephedrine hydrochloride core tablets were prepared by direct compression like example 1 process.

2. Seal coating: Aqueous dispersion of opadry AMB II white 88A180040 was sprayed onto the core tablets.

3. Extended release coating: Hydroalcoholic dispersion of Cellulose acetate with suitable coating aids (atleast plasticizer, atleast one pore-former, Acetone & purified water) was sprayed onto the seal coated tablets.

4. Sub coating: Aqueous solution of Hypromellose was sprayed onto the extended release tablets.

5. Immediate release coating (IR coating): Aqueous solution of drug and Hypromellose spray onto the sub coated tablets.

6. Film coating: Aqueous dispersion of opadry AMB II white 88A180040 was sprayed onto the IR coated tablets of step-5.

### Example 6

Cetirizine+Pseudoephedrine Non-osmotic extended release tablets (matrix tablet with HPMC K100M & 75% pseudoephedrine in core and 25% pseudoephedrine & 100% cetirizine in immediate release coating with extended release coating membrane of cellulose acetate)

| | |
|---|---|
| | Quantity/tablet |
| Ingredients | (mg) |

Core tablet

| | |
|---|---|
| Pseudoephedrine Hydrochloride | 180.000 |
| Hypromellose USP (Methocel K100M Premium DC2) | 142.875 |
| Lactose Monohydrate | 3.380 |
| Microcrystalline cellulose | 12.750 |
| Colloidal silicon dioxide | 3.000 |
| Stearic acid | 6.000 |
| Sub-total | 348.000 |

Seal coating

| | |
|---|---|
| Opadry AMB II white 88A180040 | 12.180 |
| Purified water | 48.720 |
| Sub-total | 360.185 |

Extended release coating

| | |
|---|---|
| Cellulose acetate with suitable coating aids (selected one | 28.81 |
| plasticizer, at least one pore-former, Acetone & purified | |
| water) | |
| Sub-total | 388.995 |

Sub coating

| | |
|---|---|
| Hypromellose (Methocel E3LV) | 11.670 |
| Purified water | 182.827 |
| Sub-total | 400.665 |

Immediate release drug loading

| | |
|---|---|
| Pseudoephedrine Hydrochloride | 60.000 |
| Cetirizine Hydrochloride | 10.000 |
| Hypromellose USP (Methocel E3LV) | 10.600 |
| Disodium succinate Hexahydrate | 14.627 |
| Purified water | 142.841 |
| Sub-total | 495.892 |

Film coating

| | |
|---|---|
| Opadry AMB II white 88A180040 | 19.836 |
| Purified water USP^{@} | 79.344 |
| Total | 515.728 |
| Opacode Black S-1-17823 | 0.043 |

Brief manufacturing process:

1. Pseudoephedrine hydrochloride core tablets were prepared by direct compression like example 1 process.

2. Seal coating: Aqueous dispersion of opadry AMB II white 88A180040 was sprayed onto the core tablets.

3. Extended release coating: Hydroalcoholic dispersion of Cellulose acetate was sprayed onto the seal coated tablets.

4. Sub coating: Aqueous solution of Hypromellose was sprayed onto the extended release tablets.

5. Immediate release coating (IR coating): Aqueous solution of pseudoephedrine hydrochloride, cetirizine hydrochloride and other excipients were sprayed onto the sub coated tablets.

6. Film coating: Aqueous dispersion of opadry AMB II white 88A180040 was sprayed onto the IR coated tablets of step-5.

Experimental data:

In-Vitro dissolution study:

The release profile of the pharmaceutical composition comprising pseudoephedrine hydrochloride as per the present specification was evaluated through in-vitro dissolution studies. The compositions were prepared according to the formula and process of examples 1-5 and were subjected to an in-vitro dissolution study in 900 ml of 0.001N HCl, at a temperature of 37±0.5° C. using a USP apparatus-2 (paddle) with sinker at a rotation of about 50 rpm and results are given in Table-1 below:

TABLE 1
Comparative dissolution profile:

| Time Points | % Drug Release | | | | | |
|---|---|---|---|---|---|---|
| (hour) | SUDAFED | Example 1 | Example 2 | Example 3 | Example 4 | Example 5 |
| 0.5 | 25 | — | — | — | 0 | 25 |
| 1 | 26 | 29 | 0 | 0 | 0 | 26 |
| 2 | 29 | — | — | — | 3 | 26 |
| 3 | — | 53 | 2 | 2 | | — |
| 4 | 35 | — | — | — | 19 | 29 |
| 5 | — | 83 | 17 | 12 | | — |
| 6 | 43 | — | — | — | 36 | 37 |
| 7 | — | — | 39 | 36 | | — |
| 8 | 53 | — | — | — | 51 | 51 |
| 9 | — | — | 60 | 58 | — | — |
| 10 | 63 | — | — | — | 66 | 62 |
| 11 | — | — | 77 | 72 | — | — |
| 12 | 72 | — | — | — | 75 | 71 |
| 13 | — | — | — | 81 | — | — |
| 14 | 80 | — | — | — | 82 | 77 |
| 15 | — | — | — | 86 | — | — |
| 16 | 85 | — | — | — | 86 | 82 |
| 17 | — | — | — | 89 | — | — |
| 18 | 89 | — | — | — | 90 | 86 |
| 20 | 92 | — | — | — | 91 | 88 |
| 22 | 94 | — | — | — | 93 | 90 |
| 23 | — | — | — | 95 | — | — |
| 24 | 96 | — | — | — | 95 | 92 |

The extended release pseudoephedrine hydrochloride tablets 240 mg of example-5 with an unexpected combination of a rate controlling core with an extended release coating system comprising cellulose acetate, dibutyl sebacate, and polyethylene glycol in a ratio of 1:0.15: 0.3, gave comparative dissolution profile with that of SUDAFED® 24 HOUR (pseudoephedrine hydrochloride ER tablets 240 mg).

### Examples 7-8

Non-osmotic extended release pseudoephedrine hydrochloride tablets 240 mg (matrix tablet with HPMC K100M & 75% Pseudoephedrine hydrochloride in core, 25% Pseudoephedrine in immediate release coating & extended release coating with cellulose acetate:plasticizer:pore former in different ratios)

| | |
|---|---|
| Example 7 | Example 8 |

| | |
|---|---|
| Ingredients | Quantity/tablet (mg) |

Core tablet

| | | |
|---|---|---|
| Pseudoephedrine HCI | 180 | 180 |
| Hypromellose K 100M | 157.875 | 142.875 |
| Lactose Monohydrate | 3.375 | 3.375 |
| Microcrystalline cellulose | 12.75 | 12.75 |
| Colloidal silicon dioxide | 3 | 3 |
| Stearic acid | 6 | 6 |
| Sub-total | 363 | 348 |

Seal coating

| | | |
|---|---|---|
| Opadry AMB II 88A180040 white | 12.705 | 12.18 |
| Purified Water | Q.S. | 48.72 |
| Sub-total | 375.705 | 360.18 |

Extended release coating

| | | |
|---|---|---|
| Cellulose acetate | 9.393 | 18.009 |
| Dibutyl Sebacate | 10.332 | 1.801 |
| Polyethylene glycol 400 | 10.332 | 3.602 |
| Ratio of Cellulose acetate: | 1:1.1:1.1 | 1:0.1:0.2 |
| plasticizer:pore-former | | |
| Sub-total | 405.762 | 383.592 |

Sub coating

| | | |
|---|---|---|
| Hypromellose | 12.173 | 11.508 |
| Purified Water | Q.S. | 182.827 |
| Sub-total | 417.935 | 395.1 |
| Immediate release drug loading | | |
| Pseudoephedrine HCI | 60 | 60 |
| Hypromellose | 10 | 10.6 |
| Purified water | Q.S. | QS |
| Sub-Total | 487.935 | 465.7 |

Film coating

| | | |
|---|---|---|
| Opadry AMB II 88A180040 white | 19.516 | 18.628 |
| Purified Water | Q.S. | QS |
| Total | 507.451 | 484.32 |

Brief manufacturing process:
1. Pseudoephedrine hydrochloride core tablets were prepared Examples 7-8 were prepared same as example 5.

In-vitro dissolution study:

The release profile of the pharmaceutical composition comprising pseudoephedrine HCl as per the present specification was evaluated through in-vitro dissolution studies. The composition was prepared according to the formula and process of examples 7 & 8 and was subjected to an in vitro dissolution study in 900 ml of 0.001N HCl, at a temperature of 37 0.5° C. using a USP apparatus-2 (paddle) with sinker at a rotation of about 50 rpm and results are given in Table-2 below:

TABLE 2
Comparative dissolution profile:

| | % Drug Release | |
|---|---|---|
| Time Points (hour) | Example 7 | Example 8 |
| 2 | 28 | 26 |
| 8 | 67 | 52 |
| 12 | 84 | 73 |
| 24 | 97 | 96 |
| F2 factor compared with | 67 | 67 |
| SUDAFED ® 24 HOUR | | |

Surprisingly, extended release pseudoephedrine hydrochloride tablets 240 mg of examples 7-8 with an unexpected combination of a rate controlling core with an extended release coating system comprising cellulose acetate, dibutyl sebacate, polyethylene glycol in a ratio of 1:1.1:1.1 to 1:0.1:0.2, gave comparative dissolution with that of SUDAFED®24 HOUR (Pseudoephedrine Hydrochloride ER tablets 240 mg).

Comparative Bioequivalence Data: “Reference Product” [SUDAFED® ER Tablets] Versus “Test Product” [Pseudoephedrine HCl ER Tablets].

A randomized, two-treatment, two-sequence, two-period, crossover, single-dose oral Bioequivalence study of Pseudoephedrine Hydrochloride Extended-Release tablets (Test) of Aurobindo Pharma Limited example 5 and SUDAFED® 24 HOUR (Pseudoephedrine Hydrochloride ER tablets) (Reference) of Johnson & Johnson consumer Inc., USA in healthy, adult, human subjects under fasting and fed condition. T/R (Test/Reference) ratio results are as follows:—

| | Acceptable | T/R Ratio |
|---|---|---|
| Parameters | limit | (Test - Example 5) |
| Cmax/AUCI 0-t/AUC 0-inf - Fasting | 80-125 | Within the acceptable |
| | | limit |
| Cmax/AUCI 0-t/AUC 0-inf - Fed | 80-125 | Within the acceptable |
| | | limit |

Based on above T/R ratio, it was surprisingly found that Test product of example 5 results were well within the acceptable limits and was bio-equivalent to the Reference product.

Surprisingly, extended release Pseudoephedrine hydrochloride tablets 240mg of example-5 with an unexpected combination of a rate controlling core with an extended release coating system comprising cellulose acetate, dibutyl sebacate, and polyethylene glycol in a ratio of 1:0.15:0.3, gave comparative dissolution & bioavailability profiles with that of SUDAFED® 24 HOUR (Pseudoephedrine Hydrochloride ER tablets 240 mg).

We claim:

1. A non-osmotic extended release formulation comprising pseudoephedrine or a salt thereof, comprising:
I. a core comprising: a) about 60% to 90% of pseudoephedrine or a salt thereof by total weight of pseudoephedrine or a salt thereof; b) a water soluble extended release polymer; and c) one or more other pharmaceutical excipients;
II. optional seal coat;
III. extended-release coating comprising: cellulose acetate, at least one plasticizer, at least one pore-former;
IV. optional sub coat; and
V. an immediate release coating comprising about 10% to 40% of pseudoephedrine or a salt thereof by total weight of pseudoephedrine or a salt thereof, and one or more other pharmaceutical excipients.

2. The extended release formulation of claim 1, wherein the amount of pseudoephedrine hydrochloride is present in core is about 75% and wherein the amount of pseudoephedrine hydrochloride is present in immediate release coating is about 25%.

3. The extended release formulation of claim 1, wherein the amount of pseudoephedrine hydrochloride is present in core is about 180 mg, the water soluble extended release polymer is present in an amount of about 40% to 60% of by total weight of the core tablet and the said polymer is hydroxypropylmethyl cellulose, and the amount of pseudoephedrine hydrochloride is present in immediate release coating is about 60 mg.

4. The extended release formulation of claim 1, wherein the weight ratio of cellulose acetate, plasticizer and pore former in the extended release coating is from about 1:0.1:0.1 to 1:1.5:1.5.

5. The extended release formulation of claim 1, wherein plasticizer is selected from the group comprising of dibutyl sebacate, triacetin, diethyl phthalate, tributyl sebacate, glycerin, triacetin, and triethyl citrate or mixtures thereof.

6. The extended release formulation of claim 5, wherein the plasticizer is dibutyl sebacate.

7. The extended release formulation of claim 1, wherein pore-former is selected from the group comprising of polyethylene glycol, hydroxypropylmethyl cellulose, hydroxypropyl cellulose, glucose, fructose, mannitol, sorbitol, dextran, polyvinylpyrrolidone, polypropylene glycol or mixtures thereof.

8. The extended release formulation of claim 7, wherein the pore-former is polyethylene glycol.

9. A non-osmotic extended release tablet comprising:
I. a core comprising: a) about 180 mg of pseudoephedrine hydrochloride; b) hydroxypropylmethyl cellulose; and c) one or more other pharmaceutical excipients;
II. optional seal coat;
III. extended release coating comprising: about 50% to 75% weight of cellulose acetate, and about 25% to 50% weight mixture of dibutyl sebecate and polyethylene glycol, by total weight of extended release coating composition, wherein the weight ratio of cellulose acetate, dibutyl sebecate and polyethylene glycol is from about 1:0.1:0.1 to 1:0.3:0.5;
IV. optional sub coat; and
V. an immediate release coating comprising about 60 mg of pseudoephedrine hydrochloride and hydroxypropylmethyl cellulose.

10. The extended release tablet of claim 9, wherein the amount of hydroxypropylmethyl cellulose is present in core is about 40% to 60% of by total weight of the core tablet.

11. The extended release tablet of claim 9, wherein the weight ratio of cellulose acetate, dibutyl sebecate and polyethylene glycol in the extended release coating is 1:0.15:0.3.

12. The extended release tablet of claim 9, wherein the core tablet is prepared by direct compression process.

13. The amount of drug released from the extended release tablet of claim 9, is from about 20% to 35% at 2 hours, from about 40% to 75% at 8 hours, from about 65% to 90% at 12 hours and not less than 85% at 24 hours.

14. The extended release formulation of claim 1, is used for the relief of symptoms associated with nasal congestion in adults and children 12 years of age and older.

15. The non-osmotic extended release tablet composition of claim 9, further comprising cetirizine or levocetirizine or its salt thereof in the immediate release coating.
